(19) United States
(12) Patent Application Publication
(10) Pub. No.: US 2021/0369428 A1
(43) Pub. Date: Dec. 2, 2021

(54) Hands-Free Suction Apparatus

(71) Applicant: Lindite Bekiri, Markham (CA)

(72) Inventor: Lindite Bekiri, Markham (CA)

(21) Appl. No.: 17/178,993

(22) Filed: Feb. 18, 2021

Related U.S. Application Data
(60) Provisional application No. 63/031,790, filed on May 29, 2020.

(51) Int. Cl. A61C 17/06; A61C 17/08; A61M 1/00

(52) U.S. Cl. CPC A61C 17/096; A61C 17/08; A61M 1/008; A61M 2205/583; A61M 2206/20; A61M 2206/16; A61M 1/0025

(57) ABSTRACT

An apparatus for suctioning matter expelled during treatments involving an orifice of a patient; the apparatus includes a suction head, a conduit attached to the suction head and a suction device for attaching the conduit thereto, thereby putting the suction head in communication with the suction device. The conduit includes a flexible and bendable body that is easily manipulated into different positions and supports the suction head about the orifice of the patient during treatment. Preferably, the orifice is a mouth, and the treatment is a dental treatment. In this embodiment, the apparatus provides for the hands-free suction of aerosols and/or saliva and blood splatter produced from the mouth of the patient during the dental treatment.

### CROSS-REFERENCE TO RELATED APPLICATION

The present application is related to and claims priority to U.S. Provisional Patent Application No. 63/031,790 filed May 29, 2020, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of dentistry of existing art and more specifically relates to a suction apparatus for suctioning matter expelled from a patient during dental treatment.

### RELATED ART

An aerosol or “aero-solution” is a suspension of fine solid particles or liquid droplets in air or another gas. Diseases can be spread by means of these aerosols. An airborne disease is any disease that is caused by pathogens transmitted via aerosols. These aerosols may be spread through breathing, talking, coughing, sneezing, etc. This is a well-known problem in dentistry as dental work typically produces aerosols and splatter containing saliva and blood which can contain pathogens such as bacteria, viruses, fungi, etc. This poses a health risk for the dental professional as they are typically close enough to the patient to breathe in the aerosols produced, or have splatter land on them or their clothing. This also poses a health risk for other patients and dental professionals in the dental office, as aerosols may linger in the air for hours, and the splatter may land on surfaces in the dental room, thus magnifying the potential for the spread of bacteria, viruses, fungi, etc. This health risk is of particular significance in times of epidemics and pandemics.

Attempts have been made to address the problem. Such attempts have included suction devices. However, these suction devices are bulky and awkward to work with, especially for dental hygienists who do not have assistants, as they must hold the suction device whilst they try to perform the dental treatment. Thus, a suitable solution is desired.

### SUMMARY OF THE INVENTION

In view of the foregoing disadvantages inherent in the known suction device art, the present disclosure provides a novel hands-free suction apparatus. The general purpose of the present disclosure, which will be described subsequently in greater detail, is to provide a means of suctioning matter from and around an orifice of a patient during a treatment.

An apparatus is disclosed herein. As above, the apparatus may be used for suctioning matter expelled during treatments involving an orifice of a patient. The apparatus may be configured for use with a suction device. The apparatus includes a suction head and a conduit. The suction head may include a body having a conduit attachment-end opposite a matter receiving-end. The matter receiving-end may be at least substantially open, and may be configured for selective placement about the orifice of the patient. The conduit may include a proximal end opposite a distal end and an elongated length there between. The distal end may be configured for attachment to the suction device and the proximal end may be attached to the conduit attachment-end of the suction head, thereby placing the suction head in communication with the suction device. The conduit may be configured to independently support the suction head about the orifice of the patient.

According to another embodiment, a method of using an apparatus for suctioning matter expelled during treatments involving an orifice of a patient is also disclosed herein. The method includes providing the apparatus as above; attaching the distal end of the conduit to the suction device; placing the matter receiving-end of the suction head close to the orifice of the patient; switching the suction device to an on-state; and suctioning the matter expelled from the orifice of the patient during the medical treatment.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a hands-free suction apparatus, constructed and operative according to the teachings of the present disclosure.

FIG. 1 is a perspective view of an apparatus during an ‘in-use’ condition, according to an embodiment of the disclosure.

FIG. 2 is a side perspective view of the apparatus of FIG. 1 during the ‘in-use’ condition, according to an embodiment of the present disclosure.

FIG. 3 is a side perspective view of the apparatus of FIG. 1 illustrating a suction head and a conduit attached to a suction device, and a close-up inner view of the conduit showing an inner-conduit surface including a plurality of ribs thereon, according to an embodiment of the present disclosure.

FIG. 4 is a side perspective view of the apparatus of FIG. 1 illustrating the conduit having at least one constricted section, according to another embodiment of the present disclosure.

FIG. 5 is a side perspective view of the apparatus of FIG. 1 illustrating a suction-blocking device and a state-indication means, according to an embodiment of the present disclosure.

FIG. 6 is a side perspective view of the apparatus of FIG. 5 illustrating the suction-blocking device being attached to the suction head, according to an embodiment of the present disclosure.

FIG. 7 is a flow diagram illustrating a method of using an apparatus for suctioning matter expelled during treatments involving an orifice of a patient, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a suction device and more particularly to a hands-free suction apparatus as used for suctioning matter from and around an orifice of a patient during a treatment. Particularly, the hands-free suction apparatus may provide a dental professional with a hands-free means of suctioning matter such as aerosols and saliva/blood splatter from a mouth of a patient during dental treatment.

The hands-free suction apparatus may be particularly useful for dental hygienists who often do not have assistants; however, the hands-free suction apparatus may also be used by a dentist or dental assistant. For example, the hands-free suction apparatus may be used in combination with handheld suctioning (such as with saliva ejectors) carried out by a dental assistant. Further, it should be appreciated that “treatment” or “dental treatment” involves any procedure that causes the expulsion of matter from the orifice (or mouth) of the patient. For example, the dental treatment may be adjusting/trimming dentures, nightguards, etc.

Generally disclosed is an apparatus including a tubular, flexible, bendable conduit having one end that attaches to a suction device such as a high-volume evacuator (HVE), and another end that is attached to a suction head. The suction head is placed close to, but does not touch, a patient during treatment. The apparatus may be used for suctioning, and thus at least substantially reducing, aerosols or other airborne particles produced during the treatment.

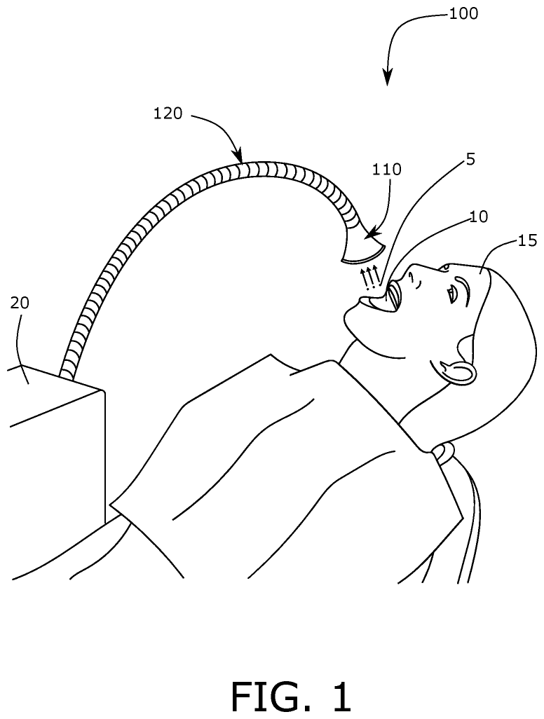

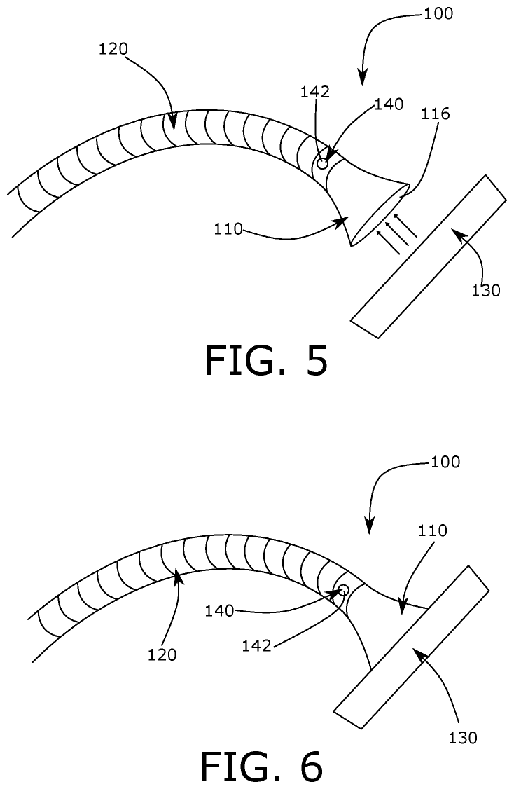

Referring now more specifically to the drawings by numerals of reference, there is shown in FIGS. 1-6, various views of an apparatus 100.

FIG. 1 shows an apparatus 100 during an ‘in-use’ condition, according to an embodiment of the present disclosure. As illustrated, the apparatus 100 may include a suction head 110 and a conduit 120. The apparatus 100 may be used for suctioning matter 5 expelled during treatments involving an orifice 10 of a patient 15. Preferably, the treatments may preferably be dental treatments. As such, the orifice 10 of the patient 15 may be a mouth of the patient 15. The apparatus 100 may be particularly useful for dental treatments whereby a dental professional such as a dentist, dental hygienist, dental nurse, etc. is on their own (thus requiring a hands-free apparatus). The matter 5 contemplated for suction by the apparatus 100 is saliva, blood, and aerosols/other airborne particles, particularly aerosols and particles containing pathogens such as viruses, bacteria, fungi, etc. Whilst use of the apparatus 100 in dental treatments is the preferred embodiment, it is also contemplated that the apparatus 100 may be utilized in other treatments.

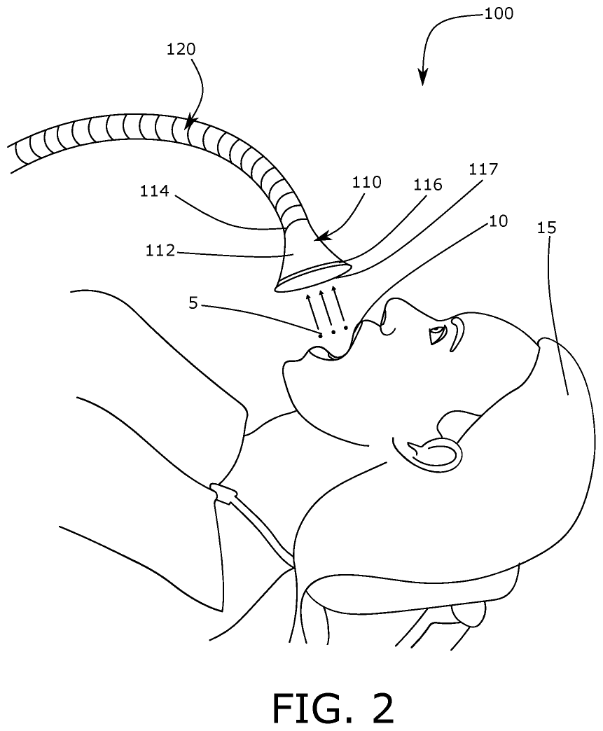

Referring now to FIGS. 2-6 showing various views of the apparatus 100 of FIG. 1, according to an embodiment of the present disclosure. The suction head 110 may include a body 112 having a conduit attachment-end 114 opposite a matter receiving-end 116. The matter receiving-end 116 may be configured for selective placement about the orifice 10 of the patient 15 and the conduit 120 may be configured to independently support the suction head 110 about the orifice 10 of the patient 15. The matter receiving-end 116 is at least substantially open to provide access to the conduit 120 (such that matter 5 is able to be suction through the matter receiving-end 116 and through the conduit 120). As shown, and as discussed above, the orifice 10 may be a mouth and the apparatus 100 may be used during dental treatments. As such, preferably, the matter receiving-end 116 may be sized to substantially conform to the mouth of the patient 15 such that the suction head 110 is able to capture as much of the matter 5 expelled there from as possible.

The matter receiving-end 116 may include a substantially circular opening 117 to effectively match an open mouth of the patient 15. Further, the suction head 110 may include a conical, or funnel-like shape. However, other sizes and shapes are also contemplated. As illustrated in FIG. 2, the suction head 110 may be placed close to the mouth of the patient 15, but not directly touching the mouth so as to not inhibit a dental professional from completing the dental treatment.

As shown, the conduit 120 may include a proximal end 122 opposite a distal end 124, an elongated length 126 there between, an outer-conduit surface 121 and an inner-conduit surface 123. The distal end 124 may be configured for attachment to the suction device 20 and the proximal end 122 may be attached to the conduit attachment-end 114 of the suction head 110, thereby placing the suction head 110 in communication with the suction device 20. The suction head 110 may be fixedly (permanently) attached to the conduit 120, or in some embodiments, the suction head 110 may be removably attached such that the suction head 110 and/or the conduit 120 may be replaced, interchanged, cleaned, etc. In some embodiments there may be a plurality of suction heads including various sizes and shapes for use with various treatments and orifices.

As above, the conduit 120 may be configured to selectively support the suction head 110 about the orifice 10 of the patient 15, thus negating a need for the apparatus 100 to be held. Preferably, the conduit 120 may comprise a bendable material having a rigidity configured to selectively support the suction head 110 in a plurality of positions. In some embodiments, the conduit 120 may include a plurality of bendable sections that are configured for selective manipulation into different positions, thereby allowing a user to position the suction head 110 in optimal positions about the orifice 10 of the patient 15, depending on treatment. For example, in the embodiment where the treatment is a dental treatment, the suction head 110 may be oriented so as to not inhibit the dental professional from working in the mouth of the patient 15, but such that the suction head 110 is able to suction as much matter 5 as possible from the mouth of the patient 15.

As above, the distal end 124 of the conduit 120 may be configured for attachment to the suction device 20 and the proximal end 122 may be attached to the conduit attachment-end 114 of the suction head 110. This thereby places the suction head 110 in communication with the suction device 20 such that the matter 5 expelled from the orifice 10 of the patient 15 may be suctioned through the matter receiving-end 116 of the suction head 110.

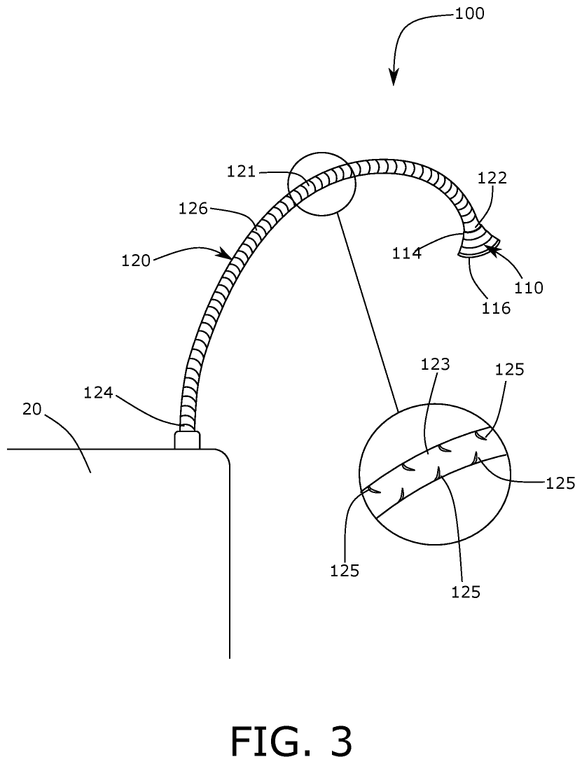

In some embodiments, to increase effectiveness of the suctioning, the inner-conduit surface 123 may include a plurality of ribs 125 staggered about the elongated length 126. FIG. 3 shows a close-up section view of the plurality of ribs 125. In the close-up view, the outer-conduit surface 121 is shown to be transparent so as to illustrate the plurality of ribs 125. The plurality of ribs 125 may be structured and arranged within the elongated length 126 (on the inner-conduit surface 123) so as to create a tornado effect within the conduit 120 when the suction device 20 is on the on-state.

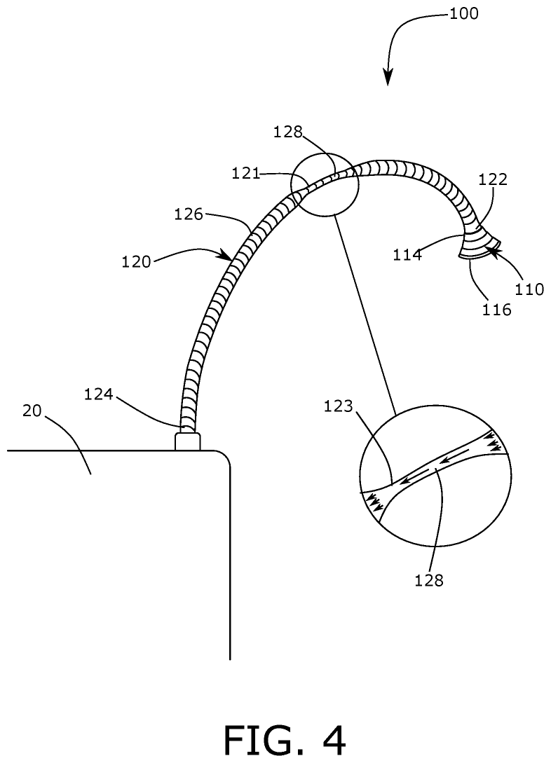

In another embodiment, as shown in FIG. 4, the conduit 120 may include at least one constricted section 128 configured to create a venturi effect within the conduit 120 when the suction device 20 is on the on-state, and thus increase effectiveness of the suctioning. Again, the outer-conduit surface 121 is shown to be transparent so as to illustrate the at least one constricted section 128. In this embodiment, the inner-conduit surface 123 may be smooth. In some embodiments, the conduit 120 may include both the plurality of ribs 125 and the at least one constricted section 128 to further increase effectiveness of the suctioning. Further, it should be appreciated that the location of the at least one constricted section 128 is shown for illustrative purposes in FIG. 4, and the at least one constricted section 128 is not limited to this location.

In some embodiments, the suction device 20 may be an existing suction device already provided where the treatments are performed—for example, a dental office for a dentist treatment. In this example, the suction device 20 may be a high-volume evacuator (HVE). Other suctions devices are also contemplated. Further, in other embodiments, the suction device 20 may be provided as a part of the apparatus 100. In this embodiment, the apparatus 100 may be provided as a kit.

As shown, the apparatus 100 may also comprise a suction-blocking device 130 configured for selective placement over the matter receiving-end 116 of the suction head 110. The suction-blocking device 130 may be a flat piece of material, such as plastic, paper, metal, etc. that includes a bigger size than an opening of the at least substantially open matter receiving-end 116. The suction-blocking device 130 may include a square shape, however other shapes are also contemplated.

Placement of the suction-blocking device 130 over the matter receiving-end 116 of the suction head 110 may temporarily inhibit matter 5 from being suctioned there through when the suction device 20 is in the on-state. The suction device 20, in the on-state, would still be suctioning, and as such, this suctioning may hold the suction-blocking device 130 to the matter receiving-end 116 of the suction head 110. The suction-blocking device 130 may be useful for if suction about the orifice 10 of the patient 15 needs to be stopped for a brief moment, so that the user does not have to power off the suction device 20.

The apparatus 100 may also include a state-indication means 140 configured to indicate a state of the suction device 20. For example, the suction device 20 may include an on-state (whereby the suction device 20 is suctioning) and an off-state (whereby the suction device 20 is not suctioning) and the state-indication means 140 may easily indicate to a user whether the suction device 20 is in the on-state or the off-state. Preferably, the state-indication means 140 may include a visual indicator such as at least one light 142. The at least one light 142 may be configured to illuminate when the suction device 20 is in the on-state, and cease illumination when the suction device 20 is in the off-state. Preferably, the at least one light 142 may be a light emitting diode (LED). Further, the at least one light 142 may be configured to emit a colored light. For example, the at least one light 142 may emit a blue or green colored light when the suction device 20 is in the on-state. In addition, the state-indication means 140 may include a sensor for sensing incoming matter 5.

As shown, in some embodiments, the state-indication means 140 may be located about the proximal end 122 of the conduit 120. Preferably, illumination of the at least one light 142 may illuminate at least a portion of the elongated length 126 of the conduit 120. In this embodiment, the conduit 120 may at least partially allow light to pass therethrough (so as to allow for illumination of the conduit 120). For example, the conduit 120 may be translucent or transparent. Further, the conduit 120 may be white in some embodiments, or may be colored in other embodiments. The state-indication means 140 is not limited to this location on the apparatus 100 however nor is it limited to being located on the apparatus 100 at all—for example, the state-indication means 140 may be an external device.

In some embodiments, the apparatus 100 may be configured for single-use. In this embodiment, the suction head 110 may comprise disposable material such as paper, plastic, polystyrene, etc. Further, the apparatus 100 may comprise recycled materials. In some embodiments, the apparatus 100 may comprise bio-degradable material. In other embodiments, the apparatus 100 may be configured for reuse. In this embodiment, the apparatus 100 may be made from materials that can be easily sterilized or disinfected. In some embodiments, the suction head 110 may be configured to suction a disinfectant solution through the apparatus 100 to disinfect the conduit 120 and the suction head 110. The apparatus 100 may be disinfected/sterilized prior to and after each patient 15. Further, in some embodiments, the apparatus 100 may include a backflow prevention means, such as a backflow prevention valve, to prevent any potential patient-to-patient contamination.

The apparatus 100 may be configured for easy portability. To aid in this, at least one of the suction head 110 and the conduit 120 may be collapsible. For example, in some embodiments, one or both of the conduit 120 and/or the suction head 110 may be selectively “stretched” into an extended position, and “collapsed” into a collapsed position. In some embodiments, at least one of the suction head 110 and the conduit 120 may be telescopic. This may allow the apparatus 100 to be used in differently sized dental offices and rooms, and also to provide a means of easily storing and transporting the apparatus 100.

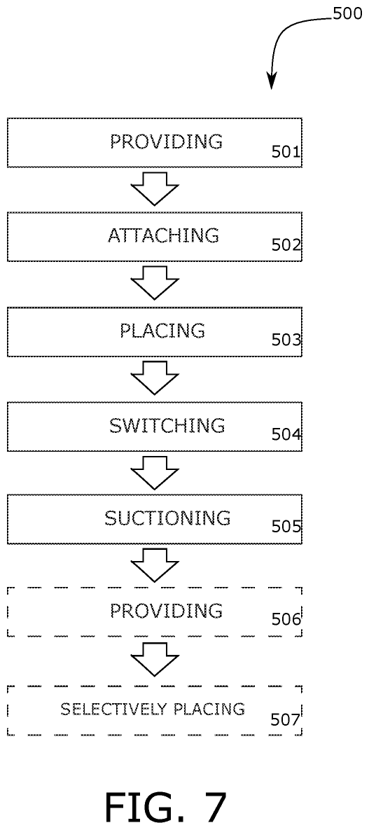

Referring now to FIG. 7 showing a flow diagram illustrating a method 500 of using an apparatus for suctioning matter expelled during treatments involving an orifice of a patient, according to an embodiment of the present disclosure. In particular, the method 500 may include one or more components or features of the apparatus 100 as described above. As illustrated, the method of use 500 may include the steps of: step one 501, providing the apparatus above; step two 502, attaching the distal end of the conduit to the suction device; step three 503, placing the matter receiving-end of the suction head close to the orifice of the patient; step four 504, switching the suction device to an on-state; and step five 505, suctioning the matter expelled from the orifice of the patient during the medical treatment. Further steps may also include step six 506, providing the apparatus further including the suction-blocking device; and step seven 507, selectively placing the suction-blocking device over the particle-receiving end of the suction head when the suction device is in the on-state, to temporarily inhibit suctioning of the matter.

It should be noted that steps six and seven (506, 507) are optional steps and may not be implemented in all cases. Optional steps of method of use 500 are illustrated using dotted lines in FIG. 7 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for suctioning matter expelled from the orifice of the patient during the treatment are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An apparatus for suctioning matter expelled during treatments involving an orifice of a patient, the apparatus configured for use with a suction device, the apparatus comprising:
a suction head including a body having a conduit attachment-end opposite a matter receiving-end, the matter receiving-end being at least substantially open, the matter receiving-end being configured for selective placement about the orifice of the patient; and
a conduit including a proximal end opposite a distal end and an elongated length there between, the distal end being configured for attachment to the suction device, the proximal end being attached to the conduit attachment-end of the suction head and thereby placing the suction head in communication with the suction device, and wherein the conduit is configured to independently support the suction head about the orifice of the patient.

2. The apparatus of claim 1, wherein the conduit comprises a bendable material having a rigidity configured to selectively support the suction head in a plurality of positions.

3. The apparatus of claim 2, wherein the conduit includes an outer-conduit surface and an inner-conduit surface, wherein the inner-conduit surface includes a plurality of ribs staggered about the elongated length, and wherein the plurality of ribs staggered about the elongated length create a tornado effect within the conduit when the suction device is in an on-state.

4. The apparatus of claim 2, wherein the conduit includes at least one constricted section, and wherein the at least one constricted section creates a venturi effect within the conduit when the suction device is in the on-state.

5. The apparatus of claim 1, further comprising a suction-blocking device for selective placement over the suction head.

6. The apparatus of claim 5, wherein the suction head is collapsible.

7. The apparatus of claim 6, wherein the particle-receiving end includes a substantially circular opening.

8. The apparatus of claim 6, wherein the conduit is collapsible.

9. The apparatus of claim 1, further comprising a state-indication means configured to indicate a state of the suction device.

10. The apparatus of claim 9, wherein the state-indication means includes at least one light.

11. The apparatus of claim 10, wherein the at least one light is configured to emit a colored light.

12. The apparatus of claim 11, wherein the at least one light is configured to illuminate when the suction device is in the on-state, and wherein the at least one light is configured to cease said illumination when the suction device is in an off-state.

13. The apparatus of claim 1, wherein the treatments are dental treatments, and wherein the orifice of the patient is a mouth of the patient.

14. An apparatus for suctioning matter expelled during dental treatments involving a mouth of a patient, the apparatus configured for use with a suction device, the apparatus comprising:
a suction head including a body having a conduit attachment-end opposite a matter receiving-end, the matter receiving-end including a substantially circular opening and being configured for selective placement about the mouth of the patient; and
a conduit including a proximal end opposite a distal end and an elongated length there between, the distal end being configured for attachment to the suction device, the proximal end being attached to the conduit attachment-end of the suction head and thereby placing the suction head in communication with the suction device, the conduit being configured to selectively support the suction head about the mouth of the patient, the conduit including a bendable material having a rigidity configured to independently support the suction head in a plurality of positions;
a state-indication means configured to indicate a state of the suction device, the state-indication means including at least one light; and
a suction-blocking device for selective placement over the suction head.

15. The apparatus of claim 14, wherein the conduit includes an outer-conduit surface and an inner-conduit surface, wherein the inner-conduit surface includes a plurality of ribs staggered about the elongated length, and wherein the plurality of ribs staggered about the elongated length create a tornado effect within the conduit when the suction device is in an on-state.

16. The apparatus of claim 14, wherein the conduit includes at least one constricted section, and wherein the at least one constricted section creates a venturi effect within the conduit when the suction device is in the on-state.

17. The apparatus of claim 14, wherein at least one of the suction head and the conduit are collapsible.

18. The apparatus of claim 14, wherein the at least one light is configured to illuminate when the suction device is in the on-state, and wherein the at least one light is configured to cease said illumination when the suction device is in an off-state.

19. A method of using an apparatus for suctioning matter expelled during treatments involving an orifice of a patient, the method comprising the steps of:
providing the apparatus including:
a suction head including a body having a conduit attachment-end opposite a matter receiving-end, the matter receiving-end being at least substantially open, the matter receiving-end being configured for selective placement about the orifice of the patient; and
a conduit including a proximal end opposite a distal end and an elongated length therebetween, the distal end being configured for attachment to a suction device, the proximal end being attached to the conduit attachment-end of the suction head and thereby placing the suction head in communication with the suction device, and wherein the conduit is configured to independently support the suction head about the orifice of the patient;
attaching the distal end of the conduit to the suction device;
placing the matter receiving-end of the suction head close to the orifice of the patient;
switching the suction device to an on-state; and
suctioning the matter expelled from the orifice of the patient during the treatment.

20. The method of claim 19, further comprising the steps of:
providing the apparatus further including a suction-blocking device; and
selectively placing the suction-blocking device over the matter-receiving end of the suction head when the suction device is in the on-state, to temporarily inhibit suctioning of the matter.
